(19) United States
(12) Patent Application Publication
(10) Pub. No.: US 2010/0016388 A1
(43) Pub. Date: Jan. 21, 2010

(54) Salts of a Selective Beta-2 Andrenoceptor Agonist

(76) Inventors: Robert Whittock, Nottinghem (GB); Jane Withnall, Leicestershire (GB)

Correspondence Address: FISH & RICHARDSON P.C., P.O BOX 1022, MINNEAPOLIS, MN, 55440-1022, US

(21) Appl. No.: 12/529,056

(22) PCT Filed: Feb. 29, 2008
(86) PCT No.: PCT/GB08/00690

§ 371 Date: Aug. 28, 2009

(30) Foreign Application Priority Data
Mar. 1, 2007 (GB) 0704000.9

(51) Int. Cl. A61K 31/428; C07D 277/62; A61P 29/00; A61P 11/00; A61P 11/08; A61P 11/06

(52) U.S. Cl. 514/367; 548/179

(57) ABSTRACT

A pharmaceutically acceptable salt of 7-[(1R)-2-({2-[3-{[2-(2-Chlorophenyl)ethyl]amino}propyl)thio]ethyl}amino)-1-hydroxyethyl]-4-hydroxy-1,3-benzothia-zol-2(3H)-one provided it is not the ditrifluoroacetate, dihydrobromide or di-acetate salt; and the use of such a compound as a medicament (for example in the treatment of respiratory diseases (such as asthma or COPD).

The present invention concerns new salt forms of 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one, compositions comprising such new salt forms, processes for preparing such salt forms, and the use of such salt forms in the treatment of disease states (such as respiratory disease states, for example asthma or COPD).

7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one free base and its ditrifluoroacetate dihydrobromide and di-acetate salts are β2 adrenoceptor agonists and are disclosed in PCT/SE2006/000981 (now published as WO2007/027134). Example 25 in PCT/SE2006/000981 produces what is referred to herein as Polymorphic Form A of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one. These compounds show at least 10-fold selectivity for β2 adrenoceptor over adrenergic α1D, adrenergic β1 and dopamine D2.

The present invention provides a pharmaceutically acceptable salt of 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one provided it is not the ditrifluoroacetate, dihydrobromide or di-acetate salt.

A pharmaceutically acceptable salt includes for example, a hydrochloride (such as a dihydrochloride), sulphate, phosphate, fumarate, maleate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate or 2-naphthalenesulphonate.

A pharmaceutically acceptable salt includes for example, a hydrochloride (such as a dihydrochloride), sulphate, phosphate, fumarate, citrate or xinafoate.

A salt of the invention can exist as a solvate (such as a hydrate), and the present invention covers all such solvents.

The polmorphic form (Polymorphic Form A) of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one has an X-ray powder diffraction (XRPD) pattern containing specific peaks at: 8.0(±0.1°), 10.0(±0.1°), 11.9(±0.1°), 16.0 (±0.1°), 18.9 (±0.1°) and 22.65(±0.1) 20.

In one particular aspect the present invention provides a polymorphic form (Polymorphic is Form B) of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one having an X-ray powder diffraction (XRPD) pattern containing specific peaks at: 7.4(±0.1°), 13.2 (±0.1°), 14.1 (±0.1°), 16.6 (±0.1°), 21.0 (±0.1°) and 21.5(±0.1°) 2θ.

In another aspect the present invention provides a process for preparing Polymorphic Form B of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one comprising adding an aqueous solution of HBr in acetonitrile to a solution of Polymorphic Form B of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one in acetonitrile and allowing the product to form as a solid.

In another aspect the present invention provides a first material form (Type A) of the dihydrochloride salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one having an X-ray powder diffraction (XRPD) pattern containing specific peaks at: 10.7(±0.1°), 11.1 (±0.1°), 13.6 (±0.1°), 20.9 (±0.1°), 22.1 (±0.1°) and 25.3(±0.1°) 20.

In another aspect the present invention provides a second material form (Type B) of the dihydrochloride salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one having an X-ray powder diffraction (XRPD) pattern containing specific peaks at: 15.2(±0.1°), 16.5 (±0.1°), 18.2 (±0.1°) and 19.0(±0.1°) 20.

In another aspect the present invention provides a third material form (Type C) of the dihydrochloride salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one having an X-ray powder diffraction (XRPD) pattern containing specific peaks at: 6.2(±0.1°), 7.4 (±0.1°), 12.5 (±0.1°), 13.2 (±0.1°), 18.6 (±0.1°) and 22.8(±0.1°) 20.

In a further aspect the present invention provides a pharmaceutically acceptable salt of 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one, for example a dihydrochloride, monoxinofoate, mono-fumarate, sulphate or mono-citrate salt.

Alternative salts of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one can be prepared by methods known in the art. For example the dihydrobromide can be treated with a base to liberate 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one, and then this can be reacted with an appropriate acid in a suitable solvent (such as an aliphatic alcohol, for example methanol) to produce the desired salt.

The salts of the present invention can be prepared by using or adapting methods presented in the Preparation or Examples below, or, by methods described in the literature.

The salts and polymorph of the invention can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay is fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoarthritis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and is peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, the present invention provides a salt as hereinbefore defined for use in therapy.

In a further aspect, the present invention provides the use of a salt as hereinbefore defined in the manufacture of a medicament for use in therapy.

In a further aspect, the present invention provides the use of a salt as hereinbefore defined is for use in the treatment of adult respiratory distress syndrome (ARDS), pulmonary emphysema, bronchitis, bronchiectasis, chronic obstructive pulmonary disease (COPD), asthma or rhinitis.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a salt as hereinbefore defined.

In particular, the compounds of this invention may be used in the treatment of adult respiratory distress syndrome (ARDS), pulmonary emphysema, bronchitis, bronchiectasis, chronic obstructive pulmonary disease (COPD), asthma and rhinitis.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The salts of the invention may be used on their own but will generally be administered in the form of a pharmaceutical composition in which the salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will for example comprise from 0.05 to 99% w (percent by weight), such as from 0.05 to 80% w, for example from 0.10 to 70% w, and such as from 0.10 to 50% w, of active ingredient, all percentages by weight being based on total composition.

The present invention also provides a pharmaceutical composition comprising a salt as hereinbefore defined, in association with a pharmaceutically acceptable adjuvant, diluent or carrier.

The invention further provides a process for the preparation of a pharmaceutical composition of the invention which comprises mixing a salt as hereinbefore defined with a pharmaceutically acceptable adjuvant, diluent or carrier.

The pharmaceutical composition may be administered topically (e.g. to the skin or to the lung and/or airways) in the form, e.g., of a cream, solution, suspension, heptafluoroalkane (HFA) aerosol or dry powder formulation, for example, a formulation in the inhaler device known as the Turbuhaler®; or systemically, e.g. by oral administration in the form of tablets, capsules, syrups, powders or granules; or by parenteral administration in the form of a solution or suspension; or by subcutaneous administration; or by rectal administration in the form of suppositories; or transdermally.

Dry powder formulations and pressurized HFA aerosols of a salt of the invention may be administered by oral or nasal inhalation. For inhalation, the compound is desirably finely divided. The finely divided compound has, for example, a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

A salt of the invention may also be administered by means of a dry powder inhaler. The inhaler may be a single or a multi dose inhaler, and may be a breath actuated dry powder inhaler.

One possibility is to mix a finely divided salt of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration a salt of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, a salt of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing a salt of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

A salt of the invention may also be administered in conjunction with other compounds used for the treatment of the above conditions.

The invention therefore further relates to combination therapies wherein a salt of the invention or a pharmaceutical composition or formulation comprising a salt of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, a salt of the invention may be combined with one of the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a salt of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a salt of the invention with a monoclonal antibody targeting B-Lymphocytes (such as CD20 (rituximab), MRA-aIL16R and T-Lymphocytes, CTLA4-Ig, HuMax 1′-15).

The present invention still further relates to the combination of a salt of the invention, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a salt of the invention, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a salt of the invention, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a salt of the invention, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 4571.5A), and BAY x 7195.

The present invention still further relates to the combination of a salt of the invention, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a salt of the invention, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a salt of the invention, and a proton pump inhibitor (such as omeprazole) or a gastroprotective histamine type 2 receptor antagonist.

The present invention further relates to the combination of a salt of the invention, and an antagonist of the histamine type 4 receptor.

The present invention still further relates to the combination of a salt of the invention, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a salt of the invention, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention further relates to the combination of a salt of the invention, and a chromone, such as sodium cromoglycate or nedocromil sodium.

The present invention still further relates to the combination of a salt of the invention, with a glucocorticoid, such as flunisolide, triamcinolone acetonide, beclomethasone dipropionate, budesonide, fluticasone propionate, ciclesonide or mometasone furoate.

The present invention further relates to the combination of a salt of the invention, with an agent that modulates a nuclear hormone receptor such as PPARs.

The present invention still further relates to the combination of a salt of the invention, together with an immunoglobulin (Ig) or Ig preparation or an antagonist or antibody modulating Ig function such as anti-IgE (for example omalizumab).

The present invention further relates to the combination of a salt of the invention, and another systemic or topically-applied anti-inflammatory agent, such as thalidomide or a derivative thereof, a retinoid, dithranol or calcipotriol.

The present invention still further relates to the combination of a salt of the invention, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a salt of the invention, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a salt of the invention, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a salt of the invention, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a salt of the invention, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a salt of the invention, together with a parenterally or topically-applied (including inhaled) local anaesthetic agent such as lignocaine or a derivative thereof.

A salt of the present invention, can also be used in combination with an anti-osteoporosis agent including a hormonal agent such as raloxifene, or a biphosphonate such as alendronate.

The present invention still further relates to the combination of a salt of the invention, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; (xxvii) inhibitor of transcription factor activation such as NFkB, API, or STATS; or, (xxviii) a glucocorticoid receptor agonist.

### General Preparative Methods

¹H NMR spectra were recorded on a Varian Inova 400 MHz or a Varian Mercury-VX 300 MHz instrument. The central peaks of chloroform-d (ε_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 8 min, 95% B1 min.

Analytical chromatography was run on a Symmetry C₁₈-column, 2.1×30 mm with 3.5 μm particle size, with acetonitrile/water/0.1% trifluoroacetic acid as mobile phase in a gradient from 5% to 95% acetonitrile over 8 minutes at a flow of 0.7 ml/min.

### Instrument Details:

XRPD (X-ray powder diffraction)—Philips X-Pert MPD machine in 0-0 configuration over the scan range 2° to 40° 2θ with 100-second exposure per 0.03° increment. The X-rays were generated by a copper long-fine focus tube operated at 45 kV and 40 mA. The wavelengths of the copper X-rays were 1.5405 Å (K_{α1}) and 1.5444 Å (K_{α2}). The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction. XRPD data are presented in the tables below, and reflection angle (° 2θ) and D-spacing (A) data (bracketed) are provided.
DSC (Differential Scanning Calorimetry) thermograms were measured using a TA Q1000 machine, with aluminium pans and pierced lids. The sample weights varied between 1 to 5 mg. The procedure was carried out under a flow of nitrogen gas (50 ml/min) and the temperature studied from 25 to 300° C. at a constant rate of temperature increase of 10° C. per minute.
TGA (Thermogravimetric Analysis) thermograms were measured using a TA Q500 machine, with platinum pans. The sample weights varied between 2 and 15 mg. The procedure was carried out under a flow of nitrogen gas (60 ml/min) and the temperature studied from 25 to 300° C. at a constant rate of temperature increase of 10° C. per minute.
¹³C CPMAS (Cross Polarisation Magic Angle Spinning) Solid State NMR spectra were obtained using a Bruker Avance 400WB machine. Samples were analysed using a 4 mm probe and under the following parameters: ramped cross polarisation, tppm 15 composite pulse, ¹H decoupling, a contact time of 2 ms, and a spin rate of 5 kHz.
Raman spectra were recorded using a Jobin Yvon Horiba Lab Ram HR raman microscope. The solid sample 0.1 mg, was placed onto a glass slide and the laser beam was focused onto a single particle that was representative of the bulk sample. Spectra were recorded as 2-4 minute acquisition over the range of 200 to 2000 cm⁻¹.
IR spectra were recorded using a Perkin Elmer Spectrum GX FT-IR System machine equipped with a Specac ATR attachment. The solid sample ˜1 mg, was placed onto the diamond surface of the ATR and a pressure of 70cN-M was applied. Spectra were recorded as 64 scans over the range of 4000 to 625 cm⁻¹, with an interval of 1 cm⁻¹ and a resolution of 4 cm⁻¹.
GVS profiles were measured using a Dynamic Vapour Sorption DVS-1 instrument. The solid sample ca. 4-10 mg was placed into a glass vessel and the weight of the sample was recorded during a dual cycle step method (40 to 90 to 0 to 90 to 0% relative humidity (RH), in steps of 10% RH).
Ion-Stoichiometry—was measured using a KOH gradient and a Dionex AS11 column with electrochemical detection and a Dionex IC3000 instrument. This technique was used for the dihydrobromide salts of Compound B only.
Chiral HPLCs were run on an Agilent 1100 LC using a Chiralcel OJ-H 250×4.6 mm column with a flow rate of 1 ml/min. Solvent A was isohexane containing 0.1% diethylamine and Solvent B was ethanol containing 0.1% diethylamine. The method was run isocratically at 20% B at a temperature of 40° C. and the run time was 31 mins. Detection was by UV absorbance at a wavelength of 220 nm.
   The abbreviations or terms used in the examples have the following meanings:
   SCX: Solid phase extraction with a sulfonic acid sorbent
   HPLC: High performance liquid chromatography

### DMF: N,N-Dimethylformamide

Compound A 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one dihydrobromide
Compound B 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one

### Preparation 1

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one Dihydrobromide

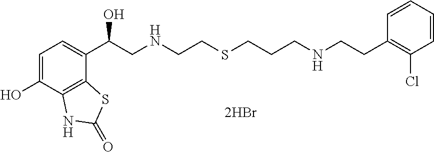

### a) 1-Chloro-2-[(E)-2-nitrovinyl]benzene

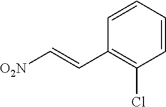

2-Chlorobenzaldehyde (ex Aldrich) (10.0 g) was mixed with nitromethane (26.05 g) and ammonium acetate (21.92 g) in acetic acid (200 mL), and the mixture was heated at reflux for 40 minutes. The mixture was allowed to cool to room temperature, and the majority of the acetic acid was removed in vacuo. The residue was dissolved in dichloromethane and washed with water, then potassium carbonate solution (×2), then water again. The organics were dried over anhydrous magnesium sulfate, filtered and evaporated to give the desired material, as an orange oil (12.83 g).

¹H NMR δ(CDCl₃) 8.41 (d, 1H), 7.62-7.57 (m, 2H), 7.52-7.48 (m, 1H), 7.43 (dt, 1H), 7.34 (ddd, 1H)

### b) 2-(2-Chlorophenyl)ethanamine

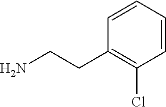

Aluminium hydride was prepared by the drop-wise addition of a solution of sulphuric acid (8.40 mL) in dry THF (60 mL) to a stirred solution of 1.0M lithium aluminium hydride in THF (314 mL), at 0-10° C., under a nitrogen atmosphere. After stirring at 5° C. for 30 minutes, a solution of 1-chloro-2-[(E)-2-nitrovinyl]benzene (12.83 g) in dry THF (160 mL) was added dropwise maintaining the internal temperature between 0° C. and 10° C. When the addition was complete the reaction was heated at reflux for 5 minutes. The mixture was allowed to cool to room temperature, then cooled to 0° C. and isopropanol (22 mL) carefully added dropwise maintaining the temperature below 20° C. 2M Sodium hydroxide (35 mL) was carefully added dropwise maintaining the temperature below 20° C. The mixture was stirred at room temperature for 30 minutes, then filtered through a layer of celite, which was then washed with THF (×3). The filtrate was evaporated to dryness. The residue was purified using silica column chromatography, using ethyl acetate to load the material, then 10% triethylamine in ethyl acetate, followed by 10% triethylamine in 45% ethanol: 45% ethyl acetate as the eluents, to give the desired material (4.66 g).

¹H NMR δ(CDCl₃) 7.36 (dd, 1H), 7.25-7.13 (m, 3H), 2.98 (dt, 2H), 2.91-2.87 (m, 2H)

### c) tert-Butyl [2-(2-chlorophenyl)ethyl]carbamate

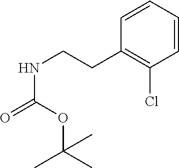

To a stirred solution of 2-(2-chlorophenyl)ethanamine (25.57 g) and triethylamine (22.87 mL) in dry THF (300 mL) was added a solution of di-tert-butyl dicarbonate (35.85 g) in dry THF (50 mL) over 10 minutes, at ambient temperature, under a nitrogen atmosphere. The reaction mixture was stirred at room temperature for 3 hours. The solvents were removed in vacuo to give the desired material, as a yellow oil (42.0 g).

¹H NMR δ(CDCL3) 7.35 (d, 1H), 7.25-7.14 (m, 3H), 4.57 (s, 1H), 3.43-3.35 (m, 2H), 2.95 (t, 2H), 1.43 (d, 9H)

### d) tert-Butyl allyl[2-(2-chlorophenyl)ethyl]carbamate

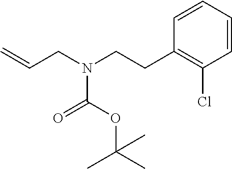

To a suspension of sodium hydride (60% in mineral oil) (7.23 g), which had been washed with ether (×3), in dry DMF (200 mL) was added a solution of tert-butyl [2-(2-chlorophenyl)ethyl]carbamate (42.0 g) in dry DMF (50 mL), over a 15 minute period, at 35° C., under a nitrogen atmosphere. When the addition was complete, the mixture was stirred at 50° C. for 90 minutes. The mixture was allowed to cool to room temperature, then allyl bromide (15.63 mL) was added slowly, keeping the temperature at 25° C., using external cooling. The mixture was stirred at room temperature for 2 hours, then diluted with water and extracted with ethyl acetate (×3). The organics were combined, washed with water, dried over anhydrous magnesium sulfate, filtered and evaporated. The residue was purified using silica column chromatography, loading with 1% ethyl acetate in isohexane, then using isohexane with ethyl acetate (0%, 1%, 2%, %5) as the eluents to give the desired material (27.0 g). There were several mixed fractions, so these were combined, and re-purified using silica column chromatography, as above, to give a further 4 g of desired material. Both crops of product were combined to give 31.0 g in total.

¹H NMR δ(CDCl₃) 7.36-7.31 (m, 1H), 7.21-7.12 (m, 3H), 5.83-5.68 (m, 1H), 5.17-5.05 (m, 2H), 3.86-3.66 (m, 2H), 3.41 (t, 2H), 3.03-2.90 (m, 2H), 1.43 (s, 9H)

HPLC: 95.90% @220 nm [M+H-Boc]+=196.1 (Calc=295.1339) (multimode+)

### e) tert-Butyl [2-(2-chlorophenyl)ethyl]{3-[(2-hydroxyethyl)thio]propyl}carbamate

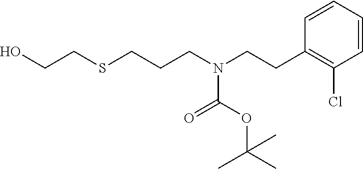

tert-Butyl allyl[2-(2-chlorophenyl)ethyl]carbamate (31.0 g) was mixed with 2-mercaptoethanol (7.37 mL), and AIBN (1.15 g), and stirred at 65° C. for 45 minutes. The mixture was cooled and more mercaptoethanol (1 mL) and AIBN (200 mg) added. The mixture was then heated at 65° C. for a further 30 minutes. The material was purified by silica column chromatography, loading the material in 20% ethyl acetate in isohexane, then eluting with 20% ethyl acetate in isohexane, changing to 50%, to give the desired material (31.94 g).

¹H NMR β_{(CDCl3)} 7.38-7.32 (m, 1H), 7.22-7.13 (m, 3H), 3.75-3.68 (m, 2H), 3.41 (t, 2H), 3.32-3.14 (m, 2H), 3.03-2.91 (m, 2H), 2.72 (t, 2H), 2.54-2.36 (m, 2H), 1.85-1.71 (m, 2H), 1.42 (s, 9H)

HPLC: 92.31% @ 220 nm [M+H-Boc]+=274.1 (Calc=373.1478) (multimode+)

### f) tert-Butyl [2-(2-chlorophenyl)ethyl]{3-[(2-oxoethyl)thio]propyl}carbamate

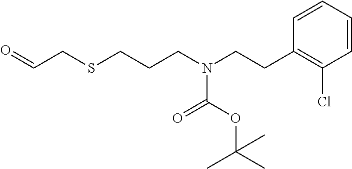

Sulfur trioxide:pyridine complex (30.52 g) was dissolved in DMSO (200 mL) and stirred at room temperature, under a nitrogen atmosphere, for 15 minutes. DCM (100 mL) was added, followed by a solution of tert-butyl [2-(2-chlorophenyl)ethyl]{3-[(2-hydroxyethyl)thio]propyl}carbamate (23.9 g) and Hunigs base (63.5 mL) in DCM (160 mL), which was added in one portion (exotherm). The resulting mixture was stirred at ambient temperature for 15 minutes. The reaction mixture was diluted with ethyl acetate, washed with water, then 1N HCl, then saturated sodium bicarbonate solution, dried over anhydrous magnesium sulfate, filtered and the solvents removed in vacuo. The material was purified by silica column chromatography eluting with 20% ethyl acetate in isohexane to give the desired material (12.43 g).

¹H NMR δ(CDCl₃) 9.46 (t, 1H), 7.36-7.32 (m, 1H), 7.21-7.13 (m, 3H), 3.40 (t, 2H), 3.29-3.13 (m, 4H), 3.02-2.90 (m, 2H), 2.45-2.34 (m, 2H), 1.82-1.69 (m, 2H), 1.49-1.36 (m, 9H)

### g) tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

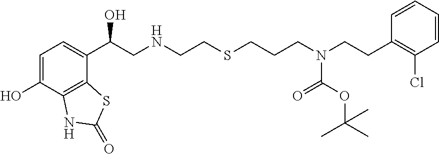

The tert-butyl[2-(2-chlorophenyl)ethyl]{3-[(2-oxoethyl)thio]propyl}carbamate (11.32 g) was dissolved in a mixture of methanol (200 mL) and acetic acid (1.74 ml). 7-[(1R)-2-amino-1-hydroxyethyl]-4-hydroxy-1,3-benzothiazol-2(3H)-one hydrochloride (8.0 g) was added to the solution, and the mixture stirred at room temperature, under a nitrogen atmosphere, for 1 hour. Sodium cyanoborohydride (1.92 g) was added and the mixture stirred for a further 2 hours. The solvents were removed in vacuo, and the residue diluted with water, basified with 0.880 aqueous ammonia, and extracted with ethyl acetate (×3) (filtered through celite during extraction). The organics were combined, washed with brine, dried over anhydrous sodium sulfate, filtered and evaporated to give a brown residue (15.5 g). The material was purified using silica column chromatography, using DCM with MeOH (2%, 5%, 10%, 20% and 30%, all with 1% 0.880 aq NH₃) as the eluent, to give the desired material (6.67 g) (38% yield)

¹H NMR 6(DMSO) 7.43-7.38 (m, 1H), 7.30-7.21 (m, 3H), 6.86 (d, 1H), 6.69 (d, 1H), 4.56 (dd, 1H), 3.23-3.10 (m, 2H), 2.88 (t, 2H), 2.71-2.48 (m, 8H), 2.46-2.39 (m, 2H), 1.72-1.62 (m, 2H), 1.40-1.22 (m, 9H)

HPLC: 97.46% @ 220 nm [M+H]+=582.1 (Calc=582.1863) (multimode+)

### h) 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one dihydrobromide

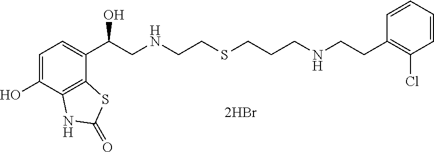

To a stirred suspension of the Boc compound from part g) (5.93 g) in DCM (20 mL) was added trifluoroacetic acid (20 mL) at 0° C., and the resulting mixture was stirred under nitrogen for 30 minutes. The mixture was diluted with toluene, and solvents removed, then azeotroped with toluene (×2). The residue was dissolved in acetonitrile, acidified with 48% aq HBr and concentrated in vacuo (not to dryness). The mixture was further diluted with acetonitrile and the precipitated solid collected by filtration, washed with acetonitrile and dried under vacuum to give 6.35 g. A 3.8% impurity was present (isomer from part e)), so the material was redissolved in a 1:1 mixture of acetonitrile:water and purified using prep HPLC (Sunfire 30×80 mm C8 column; NH₄OAc buffer; acetonitrile 5-50% over 10 minutes). The resultant material was dried overnight in a dessicator at 10 mbar over KOH and H₂SO₄. The resulting di-acetate salt was dissolved in water and basified with 0.880 aq ammonia. A white gum formed, so the aqueous was decanted off, and the gum dried in vacuo to give the free base (4.11 g). This was dissolved in hot ethanol, and the solution was filtered, then allowed to cool to room temperature. The solution was acidified with 48% aq. HBr and left to crystallize. The white solid was collected by filtration, washed with ethanol and dried in vacuo to give 3.81 g Crop 1.

¹H NMR 6(DMSO) 11.67 (s, 1H), 10.15 (s, 1H), 8.70 (s, 4H), 7.50-7.30 (m, 4H), 6.94 (d, 1H), 6.78 (d, 1H), 6.45 (s, 1H), 4.96-4.90 (m, 1H), 3.22-3.02 (m, 10H), 2.86-2.76 (m, 2H), 2.66 (t, 2H), 1.91 (quintet, 2H)

HPLC: 99.63% @ 220 nm [M+H]+=482 (calc=482.1339) (MultiMode+)

| Elemental analysis: | | C | H | N | S |
|---|---|---|---|---|---|
| Calculated: | | 41.04 | 4.70 | 6.53 | 9.96 |
| Found: | 1: | 41.07 | 4.69 | 6.67 | 9.72 |
| | 2: | 41.08 | 4.68 | 6.74 | 9.67 |
| | 3: | 40.96 | 4.68 | 6.75 | 9.67 |

The mother liquors were evaporated to dryness then triturated with acetonitrile. The solid was collected by filtration to give 719 mg Crop 2 (4.53 g total).

¹H NMR 6(DMSO) 11.67 (s, 1H), 10.15 (s, 1H), 8.80-8.60 (m, 4H), 7.50-7.29 (m, 4H), 6.94 (d, 1H), 6.78 (d, 1H), 6.45 (s, 1H), 4.96-4.89 (m, 1H), 3.22-3.00 (m, 10H), 2.85-2.76 (m, 2H), 2.66 (t, 2H), 1.90 (quintet, 2H)

HPLC: 99.20% @220 nm [M+H]+=482 (calc=482.1339) (MultiMode+)

| Elemental analysis: | | C | H | N | S |
|---|---|---|---|---|---|
| Calculated: | | 41.04 | 4.70 | 6.53 | 9.96 |
| Found: | 1: | 40.90 | 4.69 | 6.78 | 9.60 |
| | 2: | 41.01 | 4.70 | 6.83 | 9.60 |
| | 3: | 40.97 | 4.69 | 6.76 | 9.63 |

Enantiomeric purity: 97.78%

Crop 1 was analysed by XRPD and found to be partially crystalline Polymorph A. Slurrying 800 mg of crop 1 in dry ethanol (20 ml) for 9 days gave 670 mg of a highly crystalline solid identified by XPRD as Polymorph A.

HPLC: 99.04% @ 220 nm [M+H]+=482.1 (calc=482.1339) (MultiMode+)

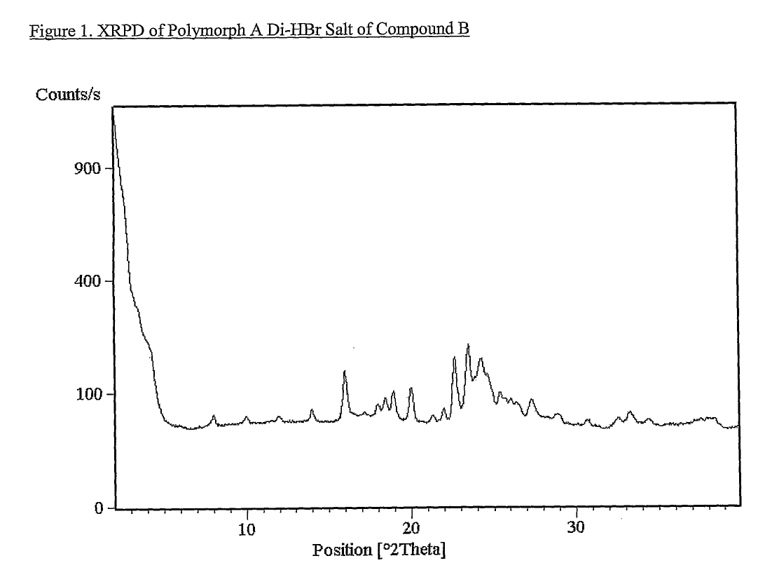

Enantiomeric purity: 98.61%

| XRPD | | | | | | | | |
|---|---|---|---|---|---|---|---|---|
| (FIG. 1.) | | | Solid State | | | | | |
| 2θ(d spacing) | | DSC | NMR | | Raman | | IR | |
| 8.0(11.1) | 27.3(3.27) | Onset = | 193.4 | 43.4 | 271.9 | 1404.8 | 3306 | 683 |
| | | 218° C. | | | | | | |
| 10.0(8.8) | 29.0(3.08) | | 184.7 | 28.2 | 328.1 | 1233.4 | 2947 | |
| 11.9(7.4) | 30.7(2.92) | | 178.0 | 25.2 | 401.0 | 1425.9 | 2784 | |
| 14.0(6.3) | 32.6(2.75) | | 174.9 | | 451.5 | 1440.5 | 1649 | |
| 16.0(5.6) | 33.3(2.69) | | 161.3 | | 462.3 | 1515.2 | 1589 | |
| 17.2(5.2) | 34.4(2.61) | | 143.0 | | 485.4 | 1573.0 | 1514 | |
| 18.0(4.93) | | | 134.2 | | 553.6 | 1590.5 | 1477 | |
| 18.5(4.81) | | | 132.2 | | 600.9 | 1651.6 | 1443 | |
| 18.9(4.69) | | | 131.2 | | 669.6 | 1700.5 | 1412 | |
| 20.0(4.44) | | | 128.4 | | 682.5 | | 1352 | |
| 21.3(4.16) | | | 124.9 | | 704.5 | | 1298 | |
| 22.03(4.03) | | | 122.7 | | 740.9 | | 1212 | |
| 22.65(3.93) | | | 120.8 | | 764.7 | | 1179 | |
| 23.5(3.79) | | | 111.0 | | 791.9 | | 1053 | |
| 24.3(3.66) | | | 78.2 | | 821.5 | | 1001 | |
| 24.7(3.61) | | | 72.6 | | 912.8 | | 931 | |
| 25.4(3.51) | | | 65.0 | | 935.7 | | 803 | |
| 25.74(3.46) | | | 52.2 | | 1037.5 | | 749 | |
| 26.0(3.42) | | | 49.4 | | 1054.7 | | 710 | |

FIG. 1. XRPD of Polymorph A Di-HBr Salt of Compound B

The following Examples illustrate the invention.

### EXAMPLE 1

### 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one Dihydrobromide—Polymorph B

### a) N-[2-(2-Chlorophenyl)ethyl]acrylamide

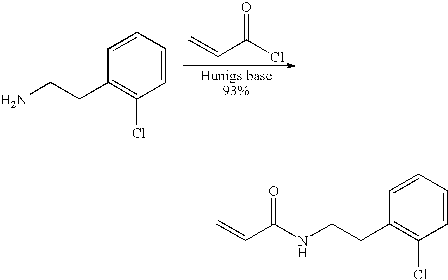

2-(2-Chlorophenyl)ethanamine (ex Aldrich) (1 eq, 491 g, 3.16 mol) was dissolved in dichloromethane (2500 ml). The solution was cooled to 0° C. and Hunigs base (1 eq, 522 ml, 3.16 mol) added. Acryloyl chloride (1 eq, 257 ml, 3.16 mol) was added dropwise, keeping the temperature between 0° C. and 5° C. throughout the 2 hour addition. The reaction was warmed to ambient temperature and stirred overnight. The mixture was diluted with dichloromethane (1500 ml) and washed with 2M HCl (2×1000 ml), then water (1×1000 ml), dried over anhydrous sodium sulfate, filtered and evaporated to give the desired material as a white, waxy solid (646 g) (97% yield).

¹H NMR δ_{(CDCl3)} 3.01 (t, J=7.1 Hz, 2H), 3.61 (q, J=6.8 Hz, 2H), 5.62 (d, J=10.6 Hz, 2H), 6.08 (dd, J=17.6, 10.6 Hz, 1H), 6.25 (d, J=17.6 Hz, 1H), 7.18-7.24 (m, 3H), 7.34-7.38 (m, 1H)

HPLC: 94.02% @ 220 nm [M+H]+=210.1 (calc=210.0685) (MultiMode+)

### b) [(3-{[2-(2-Chlorophenyl)ethyl]amino}-3-oxopropyl)thio]acetic Acid

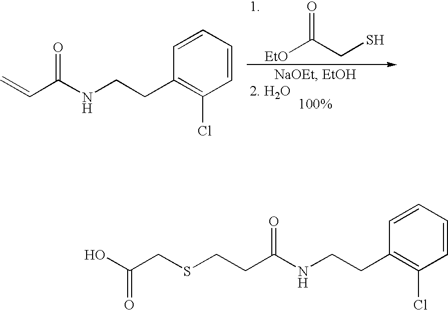

Ethyl mercaptoacetate (1 eq, 138 ml, 1.25 mol) was dissolved in ethanol (750 ml) and sodium ethoxide (21% weight in ethanol) (1 eq, 405 ml, 1.25 mol) added, keeping the internal temperature below 30° C. throughout. The reaction mixture was stirred for 1 hour before a solution of N-[2-(2-chlorophenyl)ethyl]acrylamide (1 eq, 261.8 g, 1.25 mol) in ethanol (2250 ml) was added dropwise (no increase in temperature was noted). The mixture was stirred for 18 hours. A further batch of ethyl mercaptoacetate (0.1 5 eq) and sodium ethoxide (0.15 eq) were added, and mixture stirred for a further 24 hours. A further aliquot of sodium ethoxide (20 ml) was added, followed by the slow addition of water (1000 ml), keeping the temperature below 20° C. The mixture was then stirred for 24 hours at ambient temperature. LC-MS showed complete conversion to the acid. The mixture was concentrated in vacuo to a volume of ˜litre, and another litre of water added to the mixture. The mixture was washed with tert-butyl methyl ether. The aqueous layer was acidified to pH 1 with conc. HCl, then extracted with tert-butyl methyl ether (1× 2 litre, 1×1.5 litre). The organics were combined, washed with water (1×1 litre), dried over anhydrous sodium sulfate, filtered and evaporated to give the desired material as a yellow oil (357.91 g) (95% yield).

¹H NMR δ_{(CDCl3)} 2.51 (t, J=7.6 Hz, 2H), 2.93 (t, J=7.2 Hz, 2H), 2.97 (t, J=7.2 Hz, 2H), 3.26 (s, 2H), 3.55 (q, J=6.4 Hz, 2H), 6.12 (s, 1H), 7.15-7.25 (m, 3H), 7.33-7.36 (m, 1H)

HPLC: 86.02% @ 220 nm [M+H]+=302.1 (calc=302.0617) (MultiMode+)

### c) 2-[(3-{[2-(2-Chlorophenyl)ethyl]amino}propyl)thio]ethanol

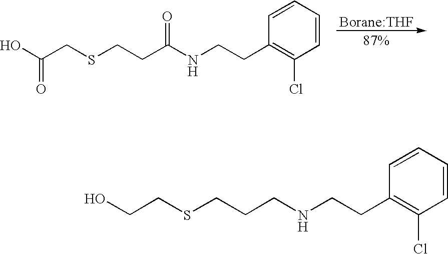

[(3-{[2-(2-Chlorophenyl)ethyl]amino}-3-oxopropyl)thio]acetic acid (107.7 g, 357 mmol) was dissolved in THF (1 litre), and a 1M borane in THF solution (ex Aldrich) (1.5 litre) was added drop-wise over ˜4 hours. The internal temperature was maintained at 30° C.±5° C. throughout the addition. The reaction was then heated to 65° C. (internal temperature) overnight, with stirring. Methanol (500 ml) was added drop-wise, followed by 2M HCl (500 ml), and the reaction refluxed gently for 4 hours. The reaction was cooled, concentrated in vacuo to a volume of ˜1 litre, and a litre of water was added to the mixture. This mixture was washed with tert-butyl methyl ether (2×500 ml). The aqueous layer was basified to ˜pH 9 with solid sodium hydroxide, then extracted with tert-butyl methyl ether (3×500 ml). The organics were combined, washed with water (1×500 ml), dried over anhydrous sodium sulfate, filtered and evaporated to give the desired material (85.3 g) (87% yield).

¹H NMR δ_{(CDCl3)} 1.73-1.82 (quintet, 2H), 1.96 (s, 1H), 2.63 (t, J=7.2 Hz, 2H), 2.72 (t, J=5.8 Hz, 2H), 2.78 (t, J=6.8 Hz, 2H), 2.83-2.97 (m, 4H), 3.74 (t, J=5.9 Hz, 2H), 7.12-7.25 (m, 3H), 7.33-7.36 (m, 1H)

HPLC: 89.70% @ 220 nm [M+H]+=274.1 (calc=274.1032) (MultiMode+)

### d) tert-Butyl [2-(2-chlorophenyl)ethyl]{3-[(2-hydroxyethyl)thio]propyl}carbamate

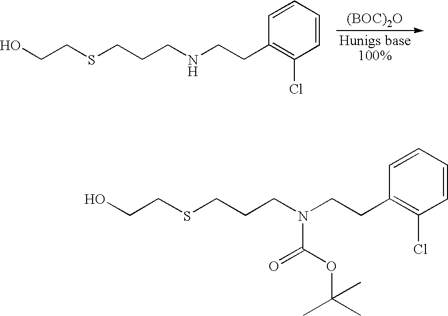

2-[(3-{[2-(2-Chlorophenyl)ethyl]amino}propyl)thio]ethanol (1 eq, 85.0 g, 312 mmol) was dissolved in dichloromethane (600 ml) and cooled in an ice bath. Hunigs base (1 eq, 51.5 ml, 312 mmol) was added, followed by a solution of di-tert-butyl dicarbonate (1 eq, 68.1 g, 312 mmol) in dichloromethane (250 ml), which was added dropwise over 2 hours to maintain an internal temperature of ˜5° C. The cooling bath was removed, and the reaction allowed to stir overnight, warming to ambient temperature. Dichloromethane (500 ml) was added and the reaction mixture was washed with water (2×500 ml), 2M HCl (2×500 ml), then water (2×500 ml) again, before being dried over anhydrous sodium sulfate, filtered and evaporated to give the desired material, as a pale, yellow oil (116 g) (100% yield).

¹H NMR δ_{(CDCl3)} 1.42 (s, 9H), 1.74-1.84 (m, 2H), 2.46-2.53 (m, 2H), 2.72 (t, J=6.7 Hz, 2H), 2.92-3.00 (m, 2H), 3.15-3.32 (m, 2H), 3.41 (t, J=7.3 Hz, 2H), 3.71 (q, J=6.0 Hz, 2H), 7.15-7.21 (m, 3H), 7.33-7.37 (m, 1H)

### e) tert-Butyl [2-(2-chlorophenyl)ethyl]{3-[(2-oxoethyl)thio]propyl}carbamate

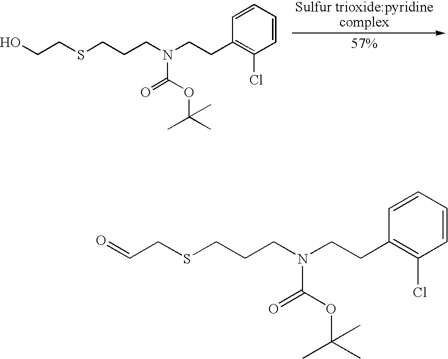

The tert-butyl [2-(2-chlorophenyl)ethyl]{3-[(2-hydroxyethyl)thio]propyl}carbamate (1 eq, 228 g, 0.61 mol) was dissolved in DMSO (1.5 litres), and treated with triethylamine (10 eq, 850 ml, 6.1 mol). The mixture was stirred vigorously, and a solution of sulfur trioxide:pyridine complex (3 eq, 291 g, 1.83 mol) in DMSO (1.5 litres) was added at such a rate that the internal temperature did not exceed 25° C. (approx 40 minutes). The reaction was poured into a mixture of ice/conc. HCl (˜4 litres, 2M), at such a rate as to keep the temperature below 30° C. This mixture was extracted with tert-butyl methyl ether (2×1.5 litres, 1×1.2 litres). The organic extracts were combined, washed with water (3×1.25 litres), dried over anhydrous magnesium sulphate, filtered and evaporated. The residue was split in to two equal portions, and each passed down a 1 kg pad of silica, eluting with isohexane:ethyl acetate (4:1) to give the desired material (130 g) (57% yield).

¹H NMR δ_{(DMSO)} 9.40 (t, J=3.5 Hz, 1H), 7.42-7.40 (m, 1H), 7.28-7.24 (m, 3H), 3.39-3.33 (m, 2H), 3.21-3.15 (m, 2H), 2.91-2.86 (m, 2H), 2.42-2.33 (m, 2H), 1.73-1.62 (m, 2H), 1.35-1.18 (m, 11H).

### f) tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate

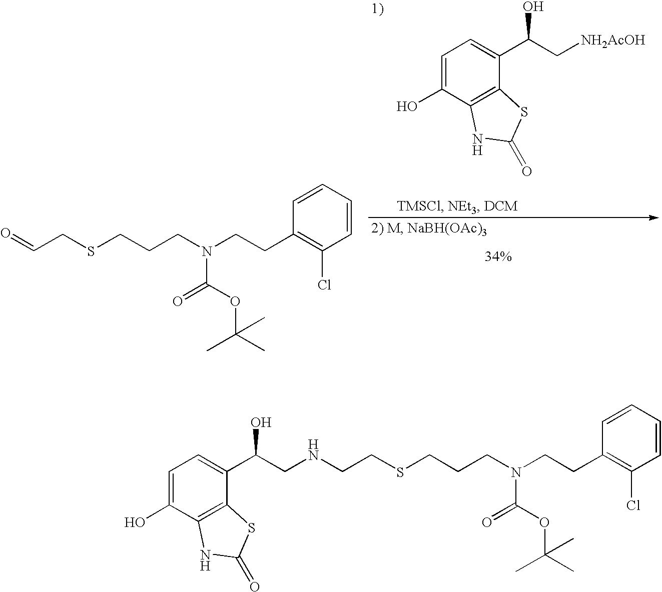

To a stirred suspension of 7-[(1R)-2-amino-1-hydroxyethyl]-4-hydroxy-1,3-benzothiazol-2(3H)-one acetate (1 eq, 92.1 g, 0.297 mol) in DCM (1200 ml) was added triethylamine (5.7 eq, 237 ml, 1.7 mol) at ambient temperature. Chlorotrimethylsilane (4.4 eq, 141 g, 164 ml, 1.3 mol) was added in portions over 20 minutes—the first 20 ml caused an exotherm to 40° C., so an ice/water bath was used to maintain the temperature at 25° C. The mixture was stirred for 4 hours at room temperature. Anhydrous magnesium sulphate (98 g) was added in a single portion to the reaction mixture, which was stirred for 15 minutes, before a is solution of tert-butyl [2-(2-chlorophenyl)ethyl]{3-[(2-oxoethyl)thio]propyl}carbamate (1.1 eq, 120 g, 0.323 mol) in DCM (800 ml) was added dropwise over 90 minutes. Sodium triacetoxyborohydride (1.2 eq, 75 g, 0.35 mol) added in one portion, maintaining the temperature at 26° C. The mixture was stirred for 16 hours at ambient temperature. Methanol (350 ml) was added in portions, followed by acetic acid (70 ml) and the mixture was stirred for 2 hours at room temperature. The solvent was removed in vacuo, and the acetic acid was removed by means of a toluene (600 ml) azeotrope. The residue was partitioned between water (1000 ml) and ethyl acetate (400 ml), the layers separated and the aqueous layer was further extracted with ethyl acetate (2×200 ml). The organics were combined and washed with water (400 ml), dried over anhydrous sodium sulfate, filtered and evaporated to give a dark oil (93 g). Isohexane (200 ml) was added and the resultant tar was manipulated with a spatula. The isohexane was decanted from the tar, and the process repeated twice more. The residue was divided into 2 batches and purified using silica column chromatography (large Biotage 75) eluting with 5% MeOH in DCM (2.5 column volumes), 10% MeOH in DCM (5 column volumes), then 16% MeOH in DCM (2.5 column volumes) to give the desired material (86.9 g) (46% yield).

¹H NMR (300 MHz, DMSO) δ 1.27-1.36 (m, 9H), 1.64-1.74 (m, 2H), 2.44-2.49 (m, 2H), 2.75-2.85 (m, 2H), 2.86-3.02 (m, 4H), 3.14-3.23 (m, 2H), 3.32-3.41 (m, 4H), 4.82 (t, J=6.1 Hz, 1H), 6.76 (d, J=8.4 Hz, 1H), 6.90 (d, J=8.4 Hz, 1H), 7.27 (s, 3H), 7.41 (d, J=7.2 Hz, 1H)

HPLC: 92.25% @ 220 nm [M+H]+=582 (calc=582.1863) (MultiMode+)

### g) 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one Dihydrobromide Polymorph B

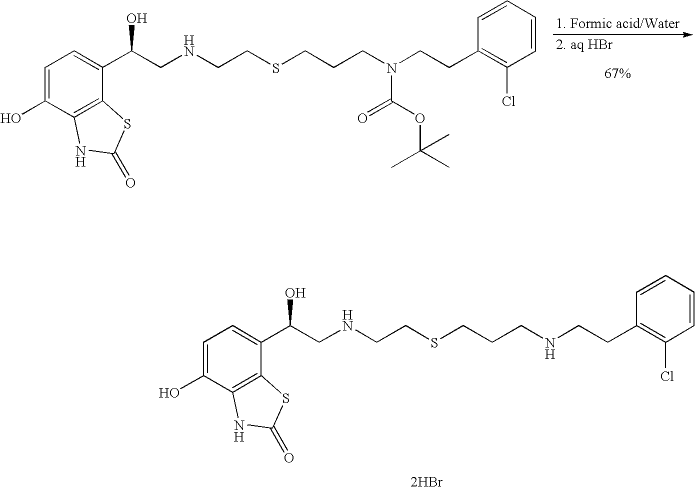

Formic acid (54 ml) was mixed with water (6 ml) and was left to age for several hours. tert-Butyl [2-(2-chlorophenyl)ethyl]{3-[(2-{[(2R)-2-hydroxy-2-(4-hydroxy-2-oxo-2,3-dihydro-1,3-benzothiazol-7-yl)ethyl]amino}ethyl)thio]propyl}carbamate (6.0 g, 10.3 mmol) was dissolved in the aqueous formic acid and stirred at room temperature for 18 hours. The solvents were removed in vacuo, and the residue was dissolved in a 4:1 mixture of acetonitrile:water (10 ml), filtered, washed with more 4:1 acetonitrile:water (4 ml), then purified using reversed phase HPLC on a 30×100 Sunfire column, injecting 2 ml (500 mg) per run, and eluting with 5-50% acetonitrile in 0.2% aqueous TFA over 8 minutes, collecting 15 ml fractions. The appropriate fractions were combined and evaporated to give 5.88 g. This material was dissolved in acetonitrile (120 ml) (sometimes a suspension was formed) and acidified with a 30% solution of 48% aq HBr in acetonitrile (25 ml). The resulting suspension was agitated, left for 15 minutes, then the solid was collected by filtration, washed with acetonitrile (×5) and dried to give the desired material (4.45 g) (67% yield) as a highly crystalline solid identified by XPRD as Polymorph B.

HPLC: 98.42% @ 220 nm [M+H]+=482.1 (calc=482.1339) (MultiMode+)

| Elemental analysis: | C | H | N | S |
|---|---|---|---|---|
| Calculated: | 41.04 | 4.70 | 6.53 | 9.96 |
| Found: | 40.81 | 4.72 | 6.73 | 10.4 |

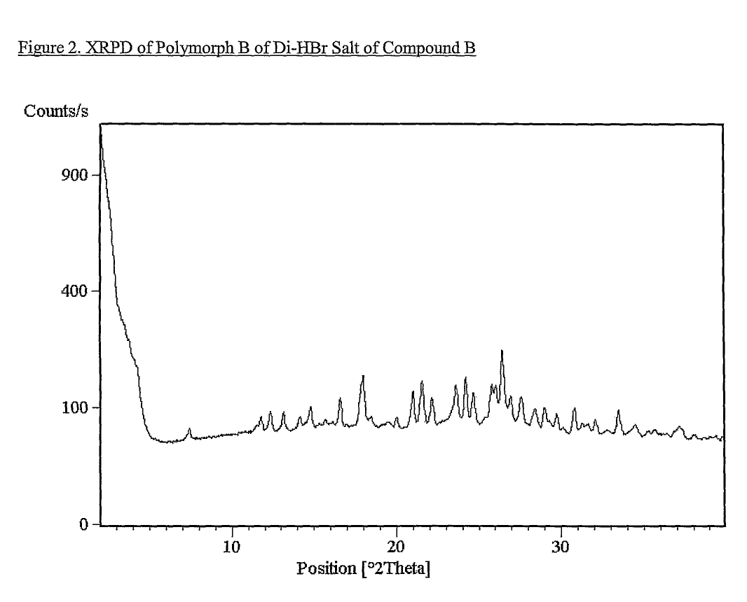

Enantiomeric purity: 97.58%

| XRPD | | | | | | | | |
|---|---|---|---|---|---|---|---|---|
| (FIG. 2.) | | | Solid State | | | | | |
| 2θ(d spacing) | | DSC | NMR | | Raman | | IR | |
| 7.4(11.9) | 27.6(3.23) | Onset = | 190.6 | 46.4 | 326.2 | 1159.7 | 2949 | 929 |
| | | 180° C. | | | | | | |
| 11.8(7.5) | 28.4(3.14) | | 179.0 | 43.9 | 381.0 | 1207.0 | 2787 | 882 |
| 12.3(7.2) | 29.0(3.08) | | 174.2 | 29.4 | 397.1 | 1221.5 | 1672 | 790 |
| 13.2(6.7) | 29.7(3.00) | | 170.6 | 25.0 | 432.1 | 1286.9 | 1652 | 753 |
| 14.1(6.3) | 30.8(2.90) | | 160.8 | | 450.5 | 1337.3 | 1626 | 708 |
| 14.8(6.0) | 33.5(2.68) | | 159.1 | | 482.2 | 1417.3 | 1588 | 682 |
| 16.6(5.3) | | | 140.8 | | 519.9 | 1588.4 | 1546 | 667 |
| 18.0(4.93) | | | 133.3 | | 541.0 | 1627.8 | 1512 | 610 |
| 20.0(4.44) | | | 129.6 | | 582.2 | 1700.5 | 1476 | |
| 21.0(4.23) | | | 125.6 | | 632.3 | | 1445 | |
| 21.5(4.13) | | | 122.1 | | 681.6 | | 1417 | |
| 22.1(4.01) | | | 120.7 | | 703.6 | | 1337 | |
| 23.6(3.77) | | | 111.0 | | 797.0 | | 1289 | |
| 24.2(3.68) | | | 109.5 | | 836.2 | | 1214 | |
| 24.6(3.61) | | | 91.1 | | 883.6 | | 1162 | |
| 25.8(3.46) | | | 69.7 | | 926.4 | | 1085 | |
| 26.0(3.42) | | | 55.7 | | 1002.3 | | 1041 | |
| 26.4(3.37) | | | 53.7 | | 1027.3 | | 980 | |
| 27.0(3.31) | | | 48.6 | | 1050.1 | | 943 | |

FIG. 2. XRPD of Polymorph B of Di-HBr Salt of Compound B

### EXAMPLE 2

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one Hydrochloride—Type A

A 37 wt/wt % solution of hydrochloric acid (175.77 μL) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was sonicated then stirred at room temperature for 16 h. The solvent was then removed in vacuo and the residue was treated with ethyl acetate (20 mL) and stirred at room temperature for 1 h. The title compound was isolated by filtration, washed with ethyl acetate (5 mL) and dried in vacuo (0.45 g).

¹H NMR (300 MHz, DMSO) δ 7.45 (m, 2H), 7.32 (m, 2H), 6.93 (d, 1H), 6.79 (d, 1H), 4.98 (m, 1H), 3.16 (m, 6H), 3.03 (m, 4H), 2.84 (t, 2H), 2.68 (t, 2H), 1.96 (m, 2H).

Enantiomeric purity: 96.7% (R); 3.3% (S).

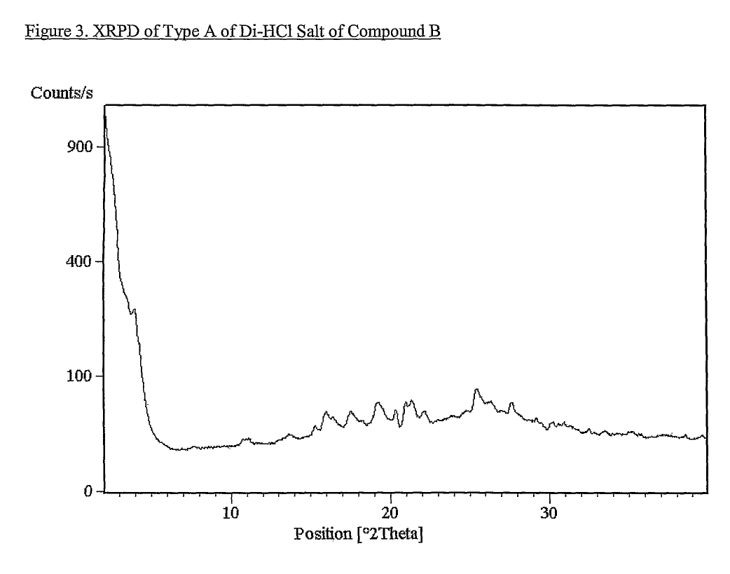

```
XRPD (FIG. 3.) 2θ(d spacing)
10.7(8.3)
11.1(8.0)
13.6(6.5)
15.3(5.8)
15.9(5.6)
17.4(5.1)
19.1(4.7)
  20.3(4.37)
  20.9(4.25)
21.32(4.17)
  22.1(4.01)
  25.3(3.51)
  26.3(3.39)
  27.0(3.30)
  27.6(3.23)
```

FIG. 3. XRPD of Type A of Di-HCl Salt of Compound B

### EXAMPLE 3

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one Hydrochloride—Type B

20 mg of Type A material (Example 2) was placed into a vial, to which was added ethanol (1 ml). The mixture was left to stir at room temperature in a capped vial for one week. The resulting suspension was then centrifuged and the solid collected and left to dry overnight in a fume hood.

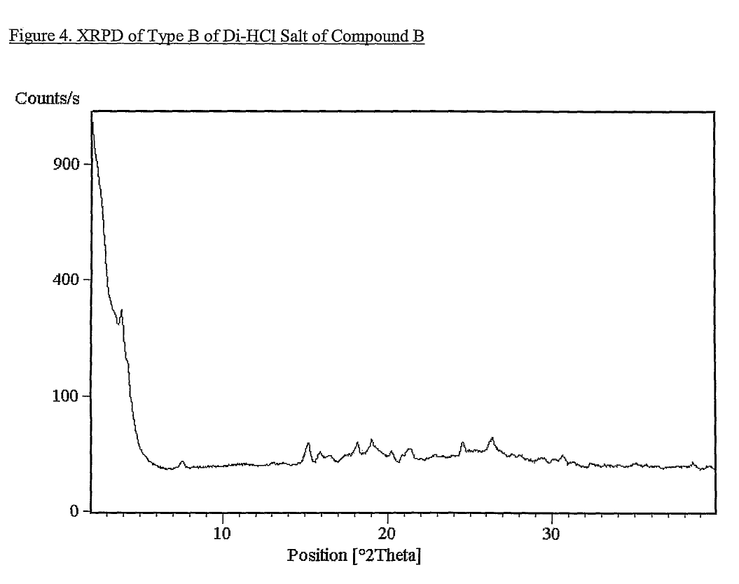

XRPD (FIG. 4.) 2θ(d spacing)
7.6(11.7)
15.2(5.8)
15.9(5.6)
16.5(5.4)
17.4(5.1)
18.2(4.88)
19.0(4.66)
20.2(4.39)
24.6(3.62)
25.3(3.52)
26.3(3.38)
27.6(3.23)
28.1(3.18)
30.6(2.92)

FIG. 4. XRPD of Type B of Di-HCl Salt of Compound B

### EXAMPLE 4

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one Hydrochloride—Type C

20 mg of Type A material (Example 2) was placed into a vial, to which was added water (1 ml). The mixture was left to stir at room temperature in a capped vial for one week. The resulting suspension was then centrifuged and the solid collected and left to dry overnight in a fume hood.

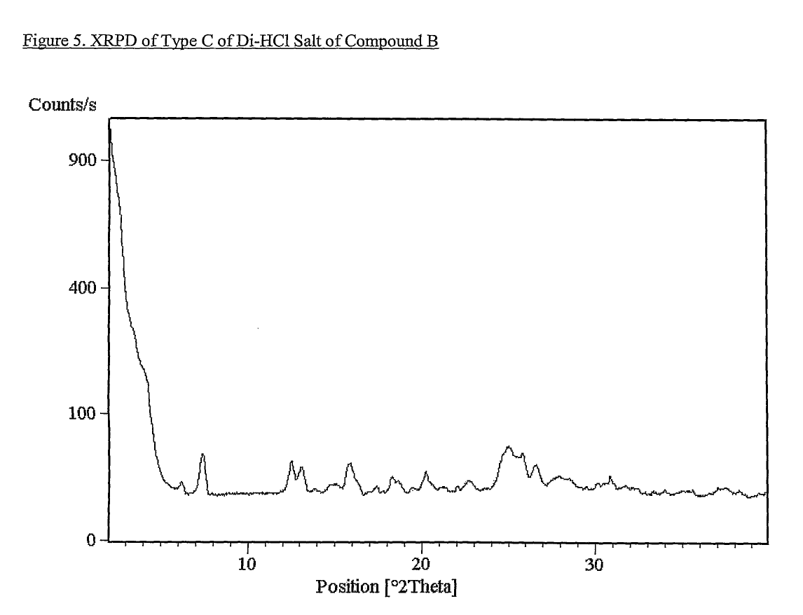

```
XRPD (FIG. 5.) 2θ(d spacing)
  6.2(14.3)
  7.4(12.0)
12.5(7.1)
13.2(6.7)
13.9(6.4)
14.7(6.0)
15.1(5.9)
15.9(5.6)
17.4(5.1)
18.2(4.86)
18.6(4.76)
20.3(4.38)
22.8(3.91)
25.8(3.45)
26.7(3.34)
30.2(2.96)
30.9(2.90)
```

FIG. 5. XRPD of Type C of Di-HCl Salt of Compound B

### EXAMPLE 5

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one monoxinafoate

1-Hydroxy-2-naphthoic Acid (394.31 mg) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was sonicated then stirred at room temperature for 16 h. The solvent was then removed in vacuo and the residue was treated with ethyl acetate (20 mL) and stirred at room temperature for 1 h. The mixture was filtered but no solid material could be isolated so the material collected on the filter and the filtrates were recombined in methanol then evaporated to dryness. The residue was stirred in diethyl ether (30 mL) for 2 h. The title compound was isolated by filtration, washed with diethyl ether (10 mL) and dried in vacuo to leave a non-crystalline product (0.53 g).

¹H NMR (300 MHz, DMSO) δ 8.20 (d, 1H), 7.74 (m, 2H), 7.48 (m, 2H), 7.40 (m, 2H), 7.33 (m, 2H), 7.04 (d, 1H), 6.93 (d, 1H), 6.78 (d, 1H), 4.95 (m, 1H), 3.16-3.00 (m, 10H), 2.83 (m, 2H), 2.66 (t, 2H), 1.93 (m, 2H). Enantiomeric purity: 97.3% (R); 2.7% (S).

Salt stoichiometry—confirmed as Mono Xinafoate salt by ¹H NMR.

### EXAMPLE 6

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one Monofumarate

Fumaric acid (120.39 mg) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was then stirred at room temperature for 2 h. The solvent was removed in vacuo and the residue was suspended in ethyl acetate (20 mL) and stirred at room temperature for 48 h. The title compound was isolated by filtration, washed with ethyl acetate (5 mL) and dried in vacuo to leave a non-crystalline product (0.59 g).

¹H NMR (300 MHz, DMSO) δ 7.43 (m, 2H), 7.31 (m, 2H), 6.91 (d, 1H), 6.76 (d, 1H), 6.55 (s, 2H), 4.84 (t, 1H), 3.07 (s, 4H), 2.96 (m, 6H), 2.74 (t, 2H), 2.62 (t, 2H), 1.90 (quintet, 2H). Enantiomeric purity: 97.2% (R); 2.8% (S).

Salt stoichiometry—confirmed as Mono Fumarate salt by ¹H NMR.

### EXAMPLE 7

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one sulfate

Concentrated sulphuric acid (510.68 μL) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was sonicated then stirred at room temperature for 2 h. The solvent was removed in vacuo and the residue was suspended in diethyl ether (20 mL) and stirred at room temperature for 1 h. The title compound was isolated by filtration, washed with diethyl ether (5 mL) and dried in vacuo to leave a non-crystalline product.

¹H NMR (300 MHz, DMSO) δ 7.45 (m, 2H), 7.32 (m, 2H), 6.93 (d, 1H), 6.77 (d, 1H), 4.95 (m, 1H), 3.50-3.00 (m, number of protons could not be determined), 2.83 (m, 2H), 2.65 (m, 2H), 1.92 (m, 2H). Enantiomeric purity: 90.5% (R); 9.5% (S).

### EXAMPLE 8

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one citrate

Citric acid (199.27 mg) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was sonicated then stirred at room temperature for 2 h. The solvent was removed in vacuo and the residue was suspended in diethyl ether (20 mL) and stirred at room temperature for 1 h. The title compound was isolated by filtration, washed with diethyl ether (5 mL) and dried in vacuo to leave a non-crystalline product.

¹H NMR (300 MHz, DMSO) δ 7.44 (m, 2H), 7.33 (m, 2H), 6.93 (d, 1H), 6.79 (d, 1H), 4.92 (m, 1H), 3.32-3.01 (m, number of protons could not be determined), 2.79 (m, 2H), 2.67-2.49 (m, number of protons could not be determined), 1.91 (m, 2H). Enantiomeric purity: 93.6% (R); 6.4% (S).

### EXAMPLE 9

### 7-[(1R)-2-({2-[(3-{[2-(2-Chlorophenyl)ethyl]amino}propyl)thio]ethyl}-amino)-1-hydroxyethyl]-4-hydroxy-1,3-benzothiazol-2(3H)-one phosphate

Phosphoric acid (119.58 mg) was added to a suspension of 7-[(1R)-2-({2-[(3-{[2-(2-chlorophenyl)ethyl]amino}propyl)thio]ethyl}-amino)-1-hydroxyethyl]-4-hydroxy-1,3-benzothiazol-2(3H)-one (0.5 g) in methanol (5 mL). The mixture was then stirred at room temperature for 1 h. The solvent was removed in vacuo and the residue was suspended in diethyl ether (20 mL) and stirred at room temperature for 16 h. The solvent had evaporated so the residue was treated with more diethyl ether (5 mL). The title compound was isolated by filtration, washed with diethyl ether (5 mL) and dried in vacuo to leave a non-crystalline product (0.47 g).

¹H NMR (300 MHz, DMSO) δ 7.44 (m, 2H), 7.31 (m, 2H), 6.92 (d, 1H), 6.76 (d, 1H), 4.93 (t, 1H), 3.17-2.91 (m, 10H), 2.88-2.56 (m, 4H), 1.95 (m, 2H) Enantiomeric purity: 93.3% (R); 6.7% (S).

### Biological Assays

Adrenergic β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds were added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. Concentration response curves for agonists were constructed and data was fitted to a four parameter logistic equation to determine both the pEC₅₀ and Intrinsic Activity. Intrinsic Activity was expressed as a fraction relative to the maximum activity determined for formoterol in each experiment.

### Selectivity Assays

### Adrenergic α1D

### Membrane Preparation

Membranes were prepared from human embryonic kidney 293 (HEK293) cells expressing recombinant human α1_{D} receptor. These were diluted in Assay Buffer (50 mM HEPES, 1 mM EDTA, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 μL of test compound (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.11 nM to 10 μM. Data was fitted to a four parameter logistic equation to determine the compound potency, which was expressed as pIC50 (negative log molar concentration inducing 50% inhibition of [³H]-prazosin binding).

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of test compound (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-0 (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean±maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding).

### Dopamine D2

### Membrane Preparation

Membranes containing recombinant human Dopamine Subtype D2s receptors were obtained from Perkin Elmer. These were diluted in Assay Buffer (50 mM HEPES, 11M EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of test compound (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.11 nM to 10 μM. Data was fitted to a four parameter logistic equation to determine the compound potency, which was expressed as pIC₅₀ (negative log molar concentration inducing 50% inhibition of [³H]-spiperone binding).

Results of the above assays are shown in Table 1 for Compound A.

TABLE 1

| | | | | β1 bind | D2 bind |
|---|---|---|---|---|---|
| Compound | β2 pEC50 | β2 Int Act | α1 bind pIC50 | p IC50 | pIC50 |
| A | 9.2 | 0.8 | 7.6 | 6.9 | 5.8 |

### Onset Assay

Dunkin-Hartley guinea-pigs (between 200 g and 300 g on delivery) were supplied by a designated breeding establishment. The guinea-pigs were killed by cervical dislocation and the trachea removed. The adherent connective tissue was removed and each trachea cut into four rings. The tissue rings were then attached to an isometric transducer. The tissues were washed and a force of 1 g was applied to each ring. In all experiments a paired curve design was used. A priming dose of 1 μM methacholine was applied to the tissues. The tissues were then washed (three times, one minute between washes), the resting tension of 1 g was reapplied and the tissues were allowed to rest for 1 hour to equilibrate. Tissues were then contracted with 1 μM methacholine and once a steady response was obtained a cumulative concentration response curve to isoprenaline (10⁻⁹ M-10⁻⁵ M) was constructed. The tissues were then washed (three times, one minute between washes) and left to rest for an hour. At the end of the resting period the tissues were contracted with 1 μM methacholine and a p[A]₅₀ concentration of test compound added. Once the tissue had reached maximum relaxation, a 30×p[A]₅₀ concentration of test compound was added. Once the tissue response had reached a plateau, 10 μM sotalol was added to the bath to confirm that the relaxation was β₂ mediated

Data were collected using the ADInstruments chart4forwindows software, which measured the maximum tension generated at each concentration of agonist.

For each concentration of the isoprenaline cumulative concentration curve, the response was calculated as % relaxation of the methacholine-induced contraction. A curve was plotted of log₁₀[agonist] (M) versus percentage inhibition of the methacholine-induced contraction. These data were then fitted to a non-linear regression curve fit. For each experiment, E/[A] curve data were fitted using a 4-parameter logistic function of the form:

$$E = β + \frac{( β - α ) \cdot {[ A ]}^{m}}{{[ A ]}^{m} + {[ A ]}_{50}^{m}}$$

E and [A] are the pharmacological effect (% relaxation) and concentration of the agonist respectively; α, β, [A]₅₀ and m are the asymptote, baseline, location and slope parameters, respectively. The p[A]₅₀ and IA of each isoprenaline curve was determined from this fit, to determine if the tissue was viable for generating an onset time for the test compounds.

For each p[A]₅₀ concentration of the test compound, the response was calculated as % relaxation of the methacholine-induced contraction. The results were plotted % relaxation against time and the time taken to reach a 90% relaxation value was calculated and recorded.

The addition of a 30×p[A]₅₀ concentration enabled determination of the maximum compound effect within the individual tissue. Hence, the % of the maximum compound effect at the p[A]₅₀ concentration was calculated and recorded.

### Pharmacokinetics in the Rat

A dose solution of the test compound was prepared using a suitable dose vehicle. The concentration of the compound in the dose solution was assayed by diluting an aliquot to a nominal concentration of 50 μg·ml⁻¹ and calibrating against duplicate injections of a standard solution and a QC standard at this concentration. Compounds were administered intravenously as a bolus into a caudal vein to groups of three 250-350 g rats (approximately 1 ml·kg⁻¹). For the oral dose, a separate group of 2 or 3 animals were dosed by oral gavage (3 ml·kg⁻¹). Delivered doses were estimated by weight loss. Food was not usually withdrawn from animals prior to dosing, although this effect was investigated if necessary.

Blood samples (0.25 ml) were taken into 1 ml syringes from the caudal vein, transferred to EDTA tubes and plasma was prepared by centrifugation (5 min at 13000 rpm) soon after sample collection, before storage at −20° C. Typical sampling times were 2, 4, 8, 15, 30, 60, 120, 180, 240, 300 (min) or until the terminal t1/2 was accurately described.

The concentration of the analyte(s) were determined in plasma by quantitative mass spectrometry. Standard and quality control stock solutions were prepared at a concentration 1 mg/ml in methanol. A range of standard and QC stocks produced by serial dilution were added to control rat plasma (50 μl). The range of concentrations covered the range of levels of analyte present in the rat samples. Standards, QCs and samples underwent liquid extraction using 50 μl of organic solvent and 100 μl of organic solvent containing an internal standard, chosen to closely resemble the analyte. The samples were then mixed by repeated inversion, stored at −20° C. for at least 1 h, and centrifuged at 3500 rpm in a centrifuge for 20 minutes. Aliquots (120 μl) of each sample were transferred for analysis using LC-MSMS. Standard and quality control samples covering the range of concentrations found in the test samples were within 25% of the nominal concentration.

Pharmacokinetic data analysis was achieved using WinNonlin. A standard non-compartmental analysis was used to estimate the parameters such as Tmax, Cmax, Lambda_z t1/2_Lambda_z, AUCall, AUCINF(observed), Cl(observed), Vss(observed).

1. A pharmaceutically acceptable salt 7-[(1R)-2-({2-[(3-{[2-(2-Chlorophenyl)ethyl]amino}propyl)thio]ethyl}amino)-1-hydroxyethyl]-4-hydroxy-1,3-benzothiazol-2(3H)-one provided it is not the ditrifluoroacetate, dihydrobromide or di-acetate salt.

2. A pharmaceutically acceptable salt as claimed in claim 1 wherein the salt is a hydrochloride, sulphate, phosphate, fumarate, maleate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate or 2-naphthalenesulphonate.

3. A pharmaceutically acceptable salt as claimed in claim 1 wherein the salt is a hydrochloride, sulphate, phosphate, fumarate, citrate or xinafoate.

4. A polymorphic form (Polymorphic Form B) of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one having an X-ray powder diffraction (XRPD) pattern containing specific peaks at: 7.4(±0.1°), 13.2 (±0.1°), 14.1 (±0.1°), 16.6 (±0.1°, 21.0 (±0.1°) and 21.5(±0.1°) 20.

5. A process for preparing Polymorphic Form B of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one as claimed in claim 3, comprising adding an aqueous solution of HBr in acetonitrile to a solution of Polymorphic Form B of the dihydrobromide salt of 7-[(1R)-2-({2-[(3-{[2-(2-Chlorophenyl)ethyl]amino}-propyl)thio]ethyl}amino)-1-hydroxyethyl]-4-hydroxy-1,3-benzothiazol-2(3H)-one in acetonitrile and allowing the product to form as a solid.

6. A pharmaceutical composition comprising a pharmaceutically acceptable salt as claimed in claim 1, or a compound as claimed in claim 4, in association with a pharmaceutically acceptable adjuvant, diluent or carrier.

7. (canceled)

8. (canceled)

9. (canceled)

10. (canceled)

11. A method of treating, or reducing the risk of, a disease or condition in which modulation of β2 adrenoreceptor activity is beneficial which comprises administering to a patient in need thereof a therapeutically effective amount of a pharmaceutically acceptable salt as claimed in claim 1, or a compound as claimed in claim 4.

12. A method of treating, or reducing the risk of, an inflammatory disease or condition which comprises administering to a patient in need thereof a therapeutically effective amount of a pharmaceutically acceptable salt as claimed in claim 1, or a compound as claimed in claim 4.

13. A method according to claim 11, wherein the disease or condition is adult respiratory distress syndrome (ARDS), pulmonary emphysema, bronchitis, bronchiectasis, chronic obstructive pulmonary disease (COPD), asthma or rhinitis.
